(19) United States
(12) Patent Application Publication
(10) Pub. No.: US 2007/0065540 A1
(43) Pub. Date: Mar. 22, 2007

(54) Enhanced steep-water

(76) Inventors: Alison M. Jones, Eden Prairie, MN (US); Ting Liu Carlson, Dayton, OH (US); Eugene Max Peters Jr, Kettering, OH (US); Michael A. Porter, Maple Grove, MN (US); Anton J. Woo, Huber Heights, OH (US); Wade W. Robey, Excelsior, MN (US); Sandra Trott, Beavercreek, OH (US)

Correspondence Address: CARGILL, INCORPORATED, LAW/24, 15407 MCGINTY ROAD WEST, WAYZATA, MN, 55391, US

(21) Appl. No.: 10/565,894

(22) PCT Filed: Jul. 26, 2004
(86) PCT No.: PCT/US04/24015

§ 371 Date: Jul. 24, 2006

Related U.S. Application Data
(60) Provisional application No. 60/490,349, filed on Jul. 24, 2003.

(51) Int. Cl. A23B 7/10; C12P 1/00

(52) U.S. Cl. 426/53; 435/41

(57) ABSTRACT

The present invention is directed to the use of corn steep liquor, which is a co-product of wet corn milling to make fermentation products, which may be used, in animal feeds and fermentation media.

### FIELD OF THE INVENTION

The present invention relates to the fermentation of steep-water, which comes from the corn wet milling process to produce a variety of enhanced steep-water products.

### BACKGROUND

Corn wet milling is the processing of corn to make cornstarch, and other corn products. Cornstarch can be utilized as is, or subsequently processed into carbohydrate and protein feedstocks. Corn kernels are sequentially steeped, and then milled and separated into their major constituent fractions. The “solubles” fraction, or light steep-water, is a product of steeping the corn, while the germ, fiber, starch, and protein fractions are products of the milling step. Steep-water provides a relatively inexpensive starting material that includes a number of nutrients that are utilized as an ingredient in animal feed and fermentation applications.

### SUMMARY

The present invention is directed to methods of making and using enhanced steep-water products, as well as the products themselves. These methods utilize steep-water, a co-product of corn wet milling. These enhanced steep-water products may be used as additives in animal feed to enhance productivity and/or reduce the cost of production, or as nutrient inputs for subsequent fermentation processes. Examples of enhanced steep-water products include steep-water with increased levels of bacteriocins, vitamins, yeast and yeast extracts, enzymes, amino acids, organic acids, direct-fed microbials and combinations thereof. Additionally, the enhanced steep-water products can be further processed to remove excess water (dried). Dried steep-water typically has less than about 10%, 8%, 7%, or 5% water. Drying can be accomplished using any method known in the art, for example spray drying, or baking.

The enhanced steep-water products described herein can be made through a variety of methods, such as by altering the conditions in the steep-water to create an environment that will allow specific populations of microorganisms to grow. These are referred to as either endogenous (naturally present in the steep-water) or exogenous (added to the steep-water). The microorganisms can be fungal, yeast, bacterial, or combinations thereof depending on the desired enhanced steep-water product.

The enhanced steep-water products can be used as a “feedstock” input in subsequent fermentations, or blended into other materials such as animal feed.

In one aspect, endogenous yeast and/or exogenous yeast are grown in light steep-water or concentrated light steep-water to obtain yeast biomass. Thereafter, the biomass may be hydrolyzed to obtain a hydrolyzed yeast biomass. The biomass or hydrolyzed biomass may be blended into animal feed as an additive to improve animal productivity, or used as a “feedstock” nutrient for subsequent fermentation processes.

During the yeast growth phase, the majority of reducing sugars and lactate are consumed for biomass and/or glycerol production. Low sugar and low lactate yeast-steep-water is useful for fermentation processes that are sensitive to lactic acid. High glycerol containing yeast-steep-water has the advantage over conventional steep-water for its freeze-thaw stability and sweetness.

Light or concentrated light steep-water also may be fermented with microorganisms, such as Lactococcus lactis under fermentation conditions of temperature, time, pH and aeration effective to produce bacteriocins. Endogenous microbes in light steep-water can also be induced to produce bacteriocins. The bacteriocin fermentation product may be included with an animal feed to improve animal productivity.

In another aspect of the invention, light or concentrated light steep-water is fermented with microorganisms under fermentation conditions of temperature, time, pH and aeration effective to produce lysine and methionione.

Another embodiment provides growing microorganisms in light or concentrated light steep-water under fermentation conditions of temperature, time, pH and aeration effective to produce direct-fed microbials (DFM) that are useful as a nutrient supplement in animal feed. After fermentation, at least some of the DFM are separated from the fermented steep-water, prior to concentration of the steep-water by evaporation, followed by addition of the separated DFM into the concentrated steep-water product.

Finally in this aspect of the invention, light or concentrated light steep-water is fermented with microorganisms under fermentation conditions of temperature, time, pH and aeration effective to produce vitamins, such as vitamin B₁₂, riboflavin, arachidonic acid, dihomo-gama-lineolenic acid, thiamine, pathotenate, and mead acid, or vitamin precursors, such as the vitamin C precursor 5-ketogluconic acid.

In another aspect of the invention the corn steep liquor product starting material may be the fermentation product of light or concentrated light steep-water. In this aspect the light or concentrated light steep-water is fermented with endogenous or exogenous lactic and/or propionic acid producing bacteria to provide an acid enriched steep-water. The steep-water is fermented with controlled pH for a time and temperature sufficient to allow the microorganisms to produce lactate. The fermentation is important because it converts residual sugar in the steep-water into organic acid, such as, for example, lactic acid. After lactic acid is made the pH of the steep-water can be lowered and thereby enhancing the stability of the steep-water. Low steep-water pH also increases the solubility of steep solids and minimizes precipitate formation during the evaporation process used to make corn steep liquor. The fermentation also is effective for reducing the sugar content in the steep-water in an amount such that browning reactions do not deleteriously affect the color of any feed to which the acid enhanced product may be added as a result of drying the feed.

In yet another aspect of the invention, the steep-water product starting material is light or heavy low phosphorous steep-water which has a phosphorous content which is not more than about 25 weight percent of the steep-water from which the low phosphorous steep-water has been made and which has not been reduced in phosphorous content. Corn gluten feed is primarily used for cattle feeding and has about four times the amount of phosphorus needed by animals for nutrition. Moreover, much of the phosphorus is in the undesirable form of phytate [myinsoitol 1,2,3,4,5,6-hexakis (dihydrogen phosphate)].

In wet milling of corn for corn starch, kernel residues remain that include corn germ, corn bran, insoluble protein, and corn solubles. The wet milling of corn includes steeping of the corn prior to breaking the corn. Most of the phosphorus in corn is in the form of an organic phosphorus-containing compound, phytate. Steeping among other things leaches phytate out of the corn into steep-water and in some instances the steep-water is used as part of the animal feed once it is evaporated to about 50% solids to form heavy steep-water or corn steep liquor. Corn steep liquor is also used as a nutrient source for various fermentation processes.

Phytate is poorly digested by monogastric animals. Ruminants, such as cattle, can digest phytate through microorganisms found in the gastrointestinal tract and hence utilize released phosphate, but excess dietary phytate and phosphate consumed by a ruminant animal will pass through its gastrointestinal tract, be excreted as manure and become environmentally damaging in areas of extensive livestock production. This is because excessive amounts of phosphorus enter the environment and the aquifer from the animal manure. A further problem with phytate is that it associates with multivalent cations which may be nutritionally needed by the animal, and thus interfere with the bioavailability of these cations to the animal.

The phosphorus reduced aspect of the invention contemplates using steep-water from wet corn milling and removing the phytate from the steep-water by mixing the steep-water with an alkaline hydroxide, such as calcium hydroxide, magnesium hydroxide; ammonium hydroxide and mixtures thereof, to convert the phytate to an alkaline metal salt and/or ammonium salt (phytin) and to precipitate the phytin in the steep-water to provide a phytin precipitated steep-water. The alkaline metal and/or ammonium hydroxide is in an amount effective to precipitate the phytate in the steep-water and to provide an alkaline metal or ammonium phytin complex or associate the divalent metal and/or ammonium ion with the phytin such that the phytin will precipitate with the calcium metal, magnesium metal and/or ammonium ions. Calcium ions, however, are a very important aspect of the invention and work better to precipitate phosphorus than other ions even when the other ions are in an environment having a high pH. The alkaline metal or ammonium ions also complex and precipitate a small amount of inorganic phosphate in steep-water. Generally the alkaline metal and/or ammonium hydroxide will be present in amount to provide a pH of greater than about 5.5 and preferably greater than about 6.0, and a Ca/P molar ratio which is effective to precipitate at least 75% and preferably 80% of the phosphorus, which ratio is at least about 1.0, preferably at least about 1.2. Thereafter the ion/phytin complex is separated from the steep-water to provide low phosphorus steep-water. After separation of the precipitated phytin from the steep-water, the low phosphorus steep-water is used as a starting material for fermentations according to the invention.

The low phosphorous steep-water may be used as a starting material for the fermentations as described above to produce organic acids, vitamins, bacteriocins, enzymes, lysine and methionine, probiotic or direct-fed microbial supplements, yeast and yeast extract, and products high in free amino acid nitrogen content. Each of the latter fermentation products then may be mixed with or used as an animal feed with low phytate content.

Finally according to the invention, the steep-water product starting material may be the low phytate steep-water that has been fermented with microorganisms, which produce lactic acid and/or propionic acid. This fermentation produces a low phytate acid enhanced steep-water starting material. The low phytate acid enhanced starting material then may be fermented as described above with yeast, lysine and methionine-forming bacteria, vitamin-forming bacteria, bacteriocin-forming bacteria, and free amino nitrogen-forming bacteria. These fermentations produce low phytate acid enhanced products which, depending on the fermentation, includes a yeast biomass or an extract of yeast, organic acids, lysine and methionine, one or more vitamin(s), one or more bacteriocins, one or more enzymes, and free amino acid products. The low phytate acid enhanced lysine and methionine product, the low phytate acid enhanced yeast biomass, and the low phytate acid enhanced enzyme product may be used as is or mixed with an animal feed. The low phytate acid enhanced yeast biomass also may be hydrolyzed and then used as is or mixed with another animal feed.

In another embodiment a method of making a yeast enhanced steep-water is provided. Such yeast enhanced steep-water is made by incubating steep-water at a temperature of from about 25° C. to about 45° C. under aerobic conditions to produce a yeast enhanced steep-water that comprises at least 70% yeast on a microbial dry weight basis. Microbial weight is understood to mean the weight of all of the yeast, fungi, and bacteria found in the enhanced steep-water after fermentation. The yeast enhanced steep water can be made by adding one or more exogenous yeast to the steep-water and/or allowing the endogenous population of yeast to become the dominant microbial population.

The invention also includes the products produced by the methods disclosed herein. For example, the yeast enhanced steep-water product can contain at least 80%, 90%, or 95% yeast on a microbial dry weight basis. In some instances the yeast enhanced steep-water product will also include at least 7-g/L acetate, 8 g/L acetate, or at least 10 g/L acetate and/or at least 1 μg/g, 2 μg/g, or 3 μg/g biotin on a dry solid basis.

The yeast enhanced steep-water may be additionally processed by lysing at least a portion of the cells to make a yeast extract. Lysing can be accomplished by incubating the yeast enhanced steep-water at a pH of from about 4.7 to 5.2 and at a temperature of from about 42° C. to about 48° C.

Another enhanced steep-water that is provided herein is an amino acid enhanced steep-water. The amino acid enhanced steep-water can be made by incubating steep-water at a temperature less than 45° C. under anaerobic conditions such that the product contains a free amino nitrogen to total nitrogen concentration of greater than 20%. In some embodiments the amino acid enhanced steep-water product is made by additionally controlling the pH at from about 4.5 to about 5.5.

Yet another enhanced steep-water product that is provided herein is a lactate enhanced steep-water. The lactate enhanced steep-water is made by incubating steep-water at a temperature from about 36° C. to about 55° C. under anaerobic conditions to produce a lactate enhanced steep-water comprising a lactate concentration of at least 180 g/Kg of steep-water solid. In some embodiments the pH is controlled at a range of from about 4.5 to about 6.0 during the incubation.

### DETAILED DESCRIPTION

I. Definitions

“Phytate” means myoinositol 1,2,3,4,5,6-hexakis dihydrogen phosphate. This compound associates with cations and forms complexes that are sometimes called phytin. We shall also describe these metal or ammonium ion/phytate associated molecules as phytin complexes.

“Corn gluten feed” is a co-product of the wet milling of corn for products such as cornstarch and corn syrup. Corn gluten feed generally includes corn germ, corn bran, corn solubles, cracked corn, and fermentation end products.

“Steep-water” includes all varieties of water that are removed from the corn after steeping. For example, one variety of steep-water is “light steep-water” which contains the soluble materials (including protein, amino acids, sugars, and phytate) originating from the corn kernel and fermentation products (mainly lactic acid and ethanol) produced from the fermentation of corn solubles during steeping. Typical light steep-water has a dry solid content of about 8-12%. Another variety of steep-water is “heavy steep-water” which is steep-water that has a dry solid content of about 50%. Finally “concentrated steep-water” is steep-water that has from about 12% dry solids to about 49% dry solids.

“Reducing Agent” means the sum of gaseous sulfur dioxide, sulfurous acid, bisulfite ions, and sulfite ions, in the steeping process.

II. Making Enhanced Steep-Water

A. Steeping Generally

The first step in the corn wet milling process is cleaning the corn. Bulk corn is cleaned on vibrating screens to remove coarse material and fine material. These screenings remove solid material from the corn kernels so that the material does not cause processing problems such as restricted water flow through the steeping process and increased steep-water viscosity.

The second step in the corn wet milling process is steeping which is the soaking of the corn in water under controlled processing conditions of temperature, time, and reducing agent concentration. During the steeping process fermentation also occurs because the naturally occurring population of microorganisms grow. These populations contain bacteria such as lactic acid bacteria, yeast, and fungus. The combination of environmental conditions and the resulting fermentation have been found to promote diffusion of the water through the tip cap of the corn kernel into the germ and endosperm. This facilitates the softening of the kernels, which allows for better separation of the components of corn.

Steeping is accomplished by putting corn into tanks and covering the corn with water. The corn and water blend is heated to about 52° C. (125° F.) and held for about 22 to about 50 hours. Steeping may be done by continuously adding dry corn at the top of the steep while continuously withdrawing steeped corn from the bottom.

Water from the steeping process accumulates corn solubles. The water is treated with a reducing agent such as sulfur dioxide to a concentration of about 0.12 to about 0.20 weight percent. The sulfur dioxide increases the rate of water diffusion into the kernel and assists in breaking down the protein-starch matrix, which is necessary for high starch yield and quality.

As water moves from one steep tank to another it is advanced from steep to steep, and the sulfur dioxide content decreases and bacterial action increases. This results in the growth of lactic acid bacteria. The lactic acid concentration is usually about 16 to about 20% (dry basis), and the sulfur dioxide (content drops to about 0.01% or less) after the water has advanced through the steeping system.

This light steep-water contains the solubles soaked out of the corn as well as products from endogenous microbial fermentation.

Optionally, steep-water can be treated to remove phosphorous using any method known in the art, such as by mixing the steep-water with Ca(OH)₂ and/or Mg(OH)₂ to precipitate the phytate in the steep-water. Precipitation of light steep-water containing solids, and phytate, that has a pH of about 4 is accomplished by mixing a sufficient amount alkaline metal hydroxide, such as lime, and/or ammonium hydroxide (at least about 0.07%, preferably about 0.07 to about 3.0%, most preferably about 0.3 to about 1.0% w/w) to raise the pH of the light steep-water to above about 5.5 and to precipitate at least about 75% of total phosphorus in steep-water as phytin and insoluble phosphate, such as calcium phosphate. The method is also effective for precipitating at least about 80% of total oxalate in the steep-water such as insoluble calcium oxalate. Generally, more than about 90% of phytate and about 10 to about 50% of inorganic phosphate are precipitated out of steep-water as the calcium salt, and more than about 90% of the oxalate is precipitated out of steep-water as calcium oxalate. The resulting steep-water containing white calcium phytate/phosphate precipitate and calcium oxalate precipitate is subjected to vacuum filtration or horizontal basket centrifugation to produce a calcium phytate and calcium oxalate product and low phosphorus steep-water.

B. Altered Steep-Water Useful for Making Enhanced Steep-Water

Enhanced steep-water products are made by fermentations that are conducted directly in the steep-water. Accordingly, the fermentations can be conducted in a steep-water that has enhanced levels of lactic and/or propionic acid, steep-water with reduced phosphorus levels, and steep-water with reduced phosphorous levels that also have enhanced levels of lactic and/or propionic acid. Types of steep-water that can be used to make enhanced steep-water are described as follows: light steep-water, heavy steep-water, steep-water with enhanced lactic acid (CSL+); corn steep-water with a reduced phosphorus content (LPCSL); corn steep-water with reduced phosphorus content which has been fermented with lactic acid bacteria (LPCSL+); and corn steep-water which has been fermented with propionic acid bacteria; corn steep-water with reduced phosphorus content which has been fermented with propionic acid bacteria (HPCSL+).

Enhanced steep-water can be generated by inoculating (i.e. exogenous microorganisms) into the water that is added to the corn at the any point in the steeping process or at any point thereafter. Thus, increasing the population of an endogenous microorganism or providing a microorganism that is not normally present in the steeping process. For example, the microorganism can be added when the concentration of the reducing agent becomes low enough to allow the microorganism population to increase.

Enhanced steep-water can also be made by altering the conditions under which the steeping actually occurs. The altered conditions will allow exogenous or endogenous microorganisms to grow.

a. Organic Acids Enhanced Steep-Water

The preservation of animal feed ingredients and/or complete feed products during storage and transport is commonly achieved by addition of organic acids, such as propionic acid, or their salts. Propionic acid, a potent mold inhibitor, can also be generated via fermentation by a few microbial genera, such as Propionibacterium. Corn steep-water, a rich source of protein and vitamins, is utilized as a component of animal feed and can serve as a fermentation feedstock for endogenous propionic acid-producing microorganisms, as well as exogenously added microbes. Consequently, fermentation of steep-water by microorganisms to produce propionic acid creates a feed component with a natural preservative present that may provide economic advantage versus the addition of chemically derived counterparts. Steep-water with elevated propionic acid levels could be used as a feedstock for microbial fermentations resistant to propionic acid, yet prone to fungal contamination. In addition, organic acids such as lactic, citric and fumaric acid are routinely added to animal feeds to reduce gastric pH, suppress pathogenic organisms and/or as readily available energy sources via the Krebs cycle. Fermentation of steep-water by organisms that would produce these acids might offer an economical, natural alternative to current practice. Finally, organic acid-rich steep-water may be augmented with additional desired acids to meet a desired commercial specification.

Steep-water can be inoculated with one or more microorganisms to produce one or more organic acids. Similarly steep-water can be incubated under conditions to allow endogenous microorganism to grow and produce organic acids. Organic acids that may be useful include citric acid, propionic acid, lactic acid, succinic acid, and the like.

Steep-water or a phosphorus reduced steep-water may be fermented for a time and at a temperature which is effective to convert carbohydrates in the steep-water into an organic acid such as lactic acid and/or propionic acid to provide an organic acid enhanced steep-water. The organic acid enhanced steep-water may be used as a nutrient source for other fermentations or combined with an animal feed to provide organic acid enhanced steep-water containing animal feed.

For example, low phosphorus steep-water may be fermented using the endogenous bacteria (or added lactic acid forming bacteria) at a temperature of at least about 45° C., preferably about 45° C. to about 55° C. for at least about 8 hours, preferably about 8 to about 48 hours to convert fermentable sugars to lactic acid. As mentioned above, the pH of the steep-water can then be allowed to decrease and the low pH and low phosphorus steep-water can be dried to about 30 to about 90% solids and mixed with other feed ingredients to make a high moisture corn gluten feed. The low pH and low phosphorus steep-water containing feed is dried to about 6 to about 15 weight percent moisture to provide the phosphorus reduced corn gluten feed of the invention having less than about 25 weight percent phosphorus than a comparable corn gluten feed containing untreated steep-water. This feed also may be pelletized. The pH stabilized, low phosphorus steep-water can be used as is or can be dried to about 30 to about 90% solids and used as a fermentation nutrient feedstock or as light steep-water. The pH stabilized, low phosphorus steep-water will have a minimal impact on the mineral metabolism of the fermentation organisms.

Furthermore, the low phosphorous steep-water can be evaporated to about 30 to about 90% solids and combined with other feedstuff to make a generic high moisture low phosphorous animal feed. The high moisture animal feed produced using the low phosphorous steep-water which has been fermented to produce lactic acid and low pH has less mold formation after 5-14 days as compared to high moisture animal feed (more than about 12 weight percent moisture) produced with low phosphorus steep-water or steep-water that has not been fermented. Endogenous and/or added lactic acid bacteria may be utilized to produce lactic acid in the steep-water.

Endogenous and/or propionic acid bacteria may be added to the steep-water prior to fermentation. One example of propionic acid bacteria that may be utilized in the process is Propionibacterium acidipropionici strain ATCC 55737. A portion of the low phosphorous steep-water may be fermented and then recombined with the remaining low phosphorus steep-water. In this aspect of the invention, an amount of steep-water is fermented such that when the fermented steep-water is recombined with the remaining steep-water and used to produce a high moisture animal feed, the feed has from about 1 to about 4 lbs. of propionate per ton of feed. The high moisture animal feed produced using low phosphorous steep-water which has been fermented to produce propionate has less mold formation after 5-14 days as compared to an animal feed produced with low phosphorous steep-water or steep-water that has not been fermented to produce propionate. The fermented low phosphorous, high propionate containing steep-water may also be held separately for other uses.

Lactic acid bacteria that may be used in the invention include Lactobeillus spp., Lactococcus spp., Leuconostoc spp. and Bacillus coagulans. Propionic acid bacteria are selected from the group consisting of Propionibacterium acidipropionici, Propionibacterium freudenreichii, Propionibacterium shennanii, Propionibacterium jensenii, and Propionibacterium thoenii. The method is effective for providing an organic acid enhanced steep-water having at least about 20% lactic acid on a dry weight basis and/or at least 10% propionic acid on a dry weight basis. In an important aspect, the organic acid enhanced steep-water includes physiologically suitable singlet organic acids and/or mixed acid constructs. The organic acid and mixed acid constructs are selected from the group consisting of formic acid, benzoic acid, lactic acid, propionic acid, acetic acid, citric acid, their salts, and mixtures thereof.

b. Yeast Enhanced Steep-Water

Yeast and yeast extracts are another widely used nutrient source for animals and fermentation microorganisms. Yeast is also a rich source of vitamins and minerals such as pantothenate, biotin, thiamine, potassium, calcium, magnesium, iron, and zinc.

Corn steep-water product may be fermented under controlled parameters to provide a yeast-enriched biomass. The yeast biomass may be used directly in animal feed or as a fermentation feedstock or may be hydrolyzed prior to further use. In accordance with the method of the invention, the steep-water is fermented with endogenous yeast or with added yeast selected from the group consisting of Saccharomyces cerevisiae, Candida utilis, Kluyveromyces marxianus and Torulaspora delbrueckii. The fermentation is conducted at a temperature of at least about 28° C. for at least about 24 hours.

The yeast enhanced steep-water can be used to generate a yeast/steep extract by causing the yeast in the steep-water to lyse open. Typical methods of lysing the yeast include heating the yeast to the point where autolysis occurs. One of ordinary skill in the art will appreciate that the temperature upon which lysis occurs depends in part upon the pH of the steep-water.

The yeast/steep extract product can be further processed by drying.

c. Bacteriocin Enhanced Steep-Water

Antimicrobial metabolites including metabolites such as organic acids and bacteriocins provide a preservative function in foods and animal feeds. Bacteriocins are antimicrobial peptides produced by many strains of lactic acid and/or other bacteria used.

Bacteriocins generally exert their anti-microbial action by interfering with the cell wall or the membrane of target organisms, either by inhibiting cell wall biosynthesis or causing membrane pore formation, subsequently resulting in death. Accordingly, one aspect of the invention provides methods of incorporating bacteriocins into foods by the direct addition of bacteriocin-producing cultures into steep-water. The resulting bacteriocin enhanced steep-water can then be added to food

Bacteriocin enhanced steep-water can be fermented under controlled parameters that are effective for inducing elevated levels of bacteriocins from endogenous bacteria by adding exogenous bacteria to the steep-water. Bacteriocins include compounds such as nicin, subtilisin and lactococcin. The bacteriocin enhanced steep-water may be used directly in animal feed. More specifically, steep-water may be fermented with endogenous bacteria or with added bacteria selected from the group consisting of Lactobacillus spp., Lactococcus spp., Pediococcus spp., and Streptococcus spp. Fermentation is conducted at a temperature within the range of 25° C. to 60° C. for 24-72 hours.

d. Vitamin Enhanced Steep-Water

Corn steep-water can be fermented under controlled parameters to increase the amount of vitamin(s) in the steep-water to provide a vitamin enhanced steep-water product. The vitamin enhanced steep-water product may be used directly in animal feed. In accordance with the method of the invention, steep-water may be fermented with endogenous microorganisms or with exogenous microorganisms selected from the group consisting of Propionibacterium shermannii, Ashbya gossypii, Eremothecium ashbyii, Bacillus spp., Gluconobacter oxidans subsp. suboxidans, Serratia marcescens, Pseudomonas denitrificans, Mortierella alpina and combinations thereof. Fermentation is conducted at a temperature of at least about 20° C. for at least about 10 hours. The method is effective for providing a steep-water having at least about 400 micrograms/Kg of steep-water solid increased vitamin(s) content compared to the starting content of the vitamin(s) in the steep-water product. Vitamins that can be produced include, but are not limited to vitamin B₁₂, riboflavin, vitamin C, biotin, and vitamin precursors such as the vitamin C precursor 5-ketogluconic acid.

e. Enzyme Enhanced Steep-Water

Steep-water may be used with endogenous and/or exogenous microorganisms to produce vitamin enhanced steep-water. Vitamin enhanced steep-water can be produced using Bacillus amyloliquefaciens, Bacillus licheniformis, Bacillus subtilis, Bacillus subtilis containing a Bacillus amyloliquefciens gene for protease, Aspergillus niger, Aspergillus oryzae, Bacillus lentus, Humicola insolens, Trichoderma longibrachiatum, Bacillus licheniformis, Bacillus licheniformis containing a Bacillus stearothermophilus gene for α-amylase, Bacillus stearothermophilus, Aspergillus niveus and mixtures thereof. The microorganisms are cultured under fermentation conditions, such as temperature, pH, aeration and time that are effective to produce enzymes, such as proteases, xylanses, amylases and phytases and/or combinations thereof. Fermentation is conducted at a temperature of at least about 28° C. for approximately 24-72 hours.

The enzyme enhanced steep-water product may be used directly in animal feed or as a fermentation feedstock.

f. Amino Acid Enhanced Steep-Water

The amino acid content of animal feeds and fermentation media can limit the growth and/or productivity of animals and microorganisms. Amino acid enhanced steep-water can be added to animal feed and/or fermentation media to increase the growth and/or productivity. Amino acid enhanced steep-water can be made via several different methods. For example, microorganisms that have been genetically engineered to over produce one or more amino acids or exogenous microorganisms that over produce one or more amino acids can be inoculated to steep-water. Free amino acid concentration in steep-water can also be increased by introducing organisms that degrade proteins to produce free amino acids.

Accordingly, steep-water may be fermented under controlled conditions to provide an increased amount of free amino nitrogen (FAN), thus making an enhanced steep-water. The FAN enhanced steep-water product may be used directly in animal feed or as a fermentation feedstock. In one embodiment of the invention, steep-water is fermented with endogenous bacteria or exogenous bacteria selected from the group consisting of Bacillus subtilis, Bacillus amyloliquefaciens, Aspergillus niger, and Aspergillus oryzae. Fermentation is conducted at a temperature of at least about 28° C., 32° C., or 38° C. for approximately 24-72 hours. The method is effective for providing an enhanced steep-water product having at least about 5-g/Kg steep-water solid free amino nitrogen.

Current agricultural practices supplement lysine and methionine to animal feeds to provide optimal growth and feed utilization, which can be a costly process for producers. Thus, feed or feed components such as enhanced steep-water, containing increased quantities of these limiting amino acids are particularly useful. Steep-water with elevated lysine or methionine may also serve as enhanced fermentation feedstocks in that growth on these feedstocks allow for increased microbial growth.

Corn steep-water can be fermented for a time and at a temperature that is effective to provide a steep-water with enhanced levels of lysine and/or methionine as compared to steep-water that has not been fermented. The lysine/methionine enhanced steep-water product may be used as a fermentation feedstock for other fermentations, or added to an animal feed to enhance its nutritive content. In accordance with the method of the invention, steep-water product may be fermented with endogenous bacteria or with exogenous bacteria that are effective for producing lysine and/or methionine such as ones selected from the group consisting of Corynebacterium glutamicum, Corynebacterium acetoacidophilum, Brevibacterium lactofermentum, Brevibacterium flavum, Brevibacterium divaricatum, and Corynebacterium lilium. Fermentation is conducted at a temperature of at least about 30° C. for at least about 48 hours. The method is effective for providing steep-water having at approximately 20-g/Kg steep-water solid lysine and/or approximately 4-g/Kg steep-water solid methionine.

g. Direct-Fed Microbial (Probiotic) Enhanced Steep-Water

Steep-water can be fermented for a time and at a temperature, which is effective to provide a steep-water with enhanced levels of beneficial organisms that can be incorporated directly into foods, such as animal feeds. Direct-fed microbial (DFM) enhanced steep-water may be used as a nutrient source for other fermentations or combined with an animal feed to provide a DFM enhanced steep-water containing animal feed. In accordance with the method of the invention, steep-water may be fermented with endogenous and/or exogenous microbes selected from the group consisting of Lactobacillus plantrum, Lactobacillus casei, Lactobacillus acidophilus, Enterococcus faecium, Bifidobacterium bifidium; Bifidobacterium thermophilum, Bifidobacterium longum, Bacillus subtillis, Saccharomyces cerevisiae, Aspergillus oryzae and mixture thereof. Fermentation is conducted at a temperature of at least about 25° C. for at least about 10 hours. Thereafter, cells can be partially removed by centrifugation, the fermented steep-water then concentrated by conventional steep-water evaporation, followed by cooling to a temperature the specific microorganism can tolerate, then the removed cells are added back to the cooled concentrated product, to maintain the viability of the DFM organisms which would have otherwise in most cases been killed during evaporation.

The following examples illustrate methods for carrying out the invention and should be understood to be illustrative of, but not limiting upon, the scope of the invention, which is defined, in the appended claims.

III. Uses of Enhanced Steep-Water

The feeds with which the enhanced steep-water products of the invention may be mixed include but are not limited to corn gluten feed, soy hulls, wheat middlings, and other cereal grain fibers, which are co-products from milling. In one embodiment, the feed ingredients are from corn and include corn bran, cracked corn, extracted cornmeal and distillers' solubles or corn processing co-products to make a high moisture corn gluten feed. Such high moisture feed will contain from about 30 to about 70 weight percent moisture. Alternatively, the feed augmented with the enhanced steep-water products may be mixed with the other fibrous feed components and then dried and pelletized to a dry feed such as a dry corn gluten feed.

Enhanced corn steep-water products may be made such that they have a greater content of lactic acid, propionic acid, yeast, free amino nitrogen (FAN), bacteriocins, vitamins, enzymes, lysine, methionine, direct-fed microbial supplements and/or combinations thereof when compared to steep-water that has not been treated to enhance production of one or more of these products.

The resulting products may be incorporated into animal feeds or used as a component in fermentation media.

Enhanced steep-water containing organic acids may be also useful as a de-icer.

### EXAMPLES

### Example 1

### Fermentation of Steep-Water at Controlled Conditions

Steep-water was taken from Cargill, Incorporated's Dayton Ohio corn wet-milling facility. The steep-water was incubated under various conditions. These conditions were: anaerobic at pH 4.2 and 180 ppm SO₂ at various temperatures; aerobic at pH 4.2 and 180 ppm SO₂ at various temperatures; anaerobic at pH 5.4 and 180 ppm SO₂ at various temperatures; aerobic at pH 5.4 and 180 ppm SO₂ at various temperatures; anaerobic at pH 4.1 and 300 ppm SO₂ at various temperatures; aerobic at pH 4.1 and 300 ppm SO₂ at various temperatures; anaerobic at pH 4.1, 180 ppm SO₂, with the addition of 5% baker's yeast (Saccharomyces cerevesiae) at 30° C.; and aerobic at pH 4.1, 180 ppm SO₂, with the addition of 5% baker's yeast at 30° C. (Saccharomyces cerevesiae).

Anaerobic conditions were achieved by incubating 75 mL of steep-water in 125 mL serum bottles with rubber stoppers. Aerobic incubation was achieved by incubating 50 mL of light steep-water in 250 mL baffled flasks shaken at 125 rpm. Adjustments to pH were made using CaCO₃. The microbial population was examined after 48 hours of incubation under a microscope. The results from visual inspection of the samples after 48 hours of incubation are provided in Table 1, below.

TABLE 1

| | | | | | | | Microbial |
|---|---|---|---|---|---|---|---|
| | | | | | | | Population |
| Condi- | Temp | | | ppm | | Baker's | after |
| tion | C. | pH | Aeration | SO₂ | CaCo₃ | Yeast | 48 hours |
| 1 | 47 | 4.2 | An- | 180 | 0 | 0 | Long chain |
| | | | aerobic | | | | bacteria |
| 2 | 47 | 4.2 | Aerobic | 180 | 0 | 0 | Short rod |
| | | | | | | | bacteria |
| 3 | 35 | 4.2 | An- | 180 | 0 | 0 | Half yeast, |
| | | | aerobic | | | | half |
| | | | | | | | bacteria |
| 4 | 35 | 4.2 | Aerobic | 180 | 0 | 0 | Mostly |
| | | | | | | | yeast |
| 5 | 30 | 4.2 | An- | 180 | 0 | 0 | Mostly |
| | | | aerobic | | | | yeast, |
| | | | | | | | some |
| | | | | | | | bacteria |
| 6 | 30 | 4.2 | Aerobic | 180 | 0 | 0 | All yeast |
| 7 | 54 | 4.2 | An- | 180 | 0 | 0 | Long chain |
| | | | aerobic | | | | bacteria |
| 8 | 54 | 4.2 | Aerobic | 180 | 0 | 0 | Few long |
| | | | | | | | chain |
| | | | | | | | bacteria |
| 9 | 47 | 5.4 | An- | 180 | 1% | 0 | Skinny |
| | | | aerobic | | | | stringy |
| | | | | | | | rod shaped |
| | | | | | | | bacteria |
| 10 | 47 | 5.4 | Aerobic | 180 | 1% | 0 | Short |
| | | | | | | | coccid/rods |
| 11 | 35 | 5.4 | An- | 180 | 1% | 0 | Few yeast, |
| | | | aerobic | | | | mostly |
| | | | | | | | bacteria |
| 12 | 35 | 5.4 | Aerobic | 180 | 1% | 0 | All yeast |
| 13 | 30 | 5.4 | An- | 180 | 1% | 0 | Half yeast, |
| | | | aerobic | | | | half |
| | | | | | | | bacteria |
| 14 | 30 | 5.4 | Aerobic | 180 | 1% | 0 | Mostly |
| | | | | | | | yeast |
| 15 | 54 | 5.4 | An- | 180 | 1% | 0 | Long chain |
| | | | aerobic | | | | bacteria, |
| | | | | | | | a few yeast |
| 16 | 54 | 5.4 | Aerobic | 180 | 1% | 0 | Long chain |
| | | | | | | | bacteria |
| 17 | 47 | 4.2 | An- | 300 | 0% | 0 | Long chain |
| | | | aerobic | | | | bacteria |
| 18 | 47 | 4.2 | Aerobic | 300 | 0% | 0 | Shorter |
| | | | | | | | chain |
| | | | | | | | bacteria |
| 19 | 35 | 4.2 | An- | 300 | 0% | 0 | Half yeast, |
| | | | aerobic | | | | half |
| | | | | | | | bacteria |
| 20 | 35 | 4.2 | Aerobic | 300 | 0% | 0 | All yeast |
| 21 | 30 | 4.2 | An- | 300 | 0% | 0 | Mostly |
| | | | aerobic | | | | yeast, |
| | | | | | | | some rods |
| 22 | 30 | 4.2 | Aerobic | 300 | 0% | 0 | Mostly |
| | | | | | | | yeast |
| 23 | 54 | 4.2 | An- | 300 | 0% | 0 | Long chain |
| | | | aerobic | | | | bacteria |
| 24 | 54 | 4.2 | Aerobic | 300 | 0% | 0 | Very few |
| | | | | | | | long chain |
| | | | | | | | bacteria |
| 25 | 30 | 4.2 | An- | 180 | 0% | 5% | Yeast and |
| | | | aerobic | | | | bacteria |
| 26 | 30 | 4.2 | Aerobic | 180 | 0% | 5% | Yeast, |
| | | | | | | | round and |
| | | | | | | | long |
| | | | | | | | yeasts |

The results provided above show that the population of yeast can be enhanced by incubating the steep-water at temperatures from about 25° C. to about 40° C., or more specifically from about 30° C. to about 35° C. At these temperatures yeast can be selected for in either anaerobic environments or aerobic environments, however, a greater amount of yeast is achievable when aerobic conditions are used.

Sugar and organic acid profiles, ion profiles, phytate and free phosphate concentrations, total nitrogen and free amino nitrogen concentrations, and pH were measured for each sample taken at time 0, 24 hours, 48 hours, and 72 hours. The final samples (after 72 hours of incubation) were also sent to independent laboratories for lysine, methionine, and biotin analysis.

A. Amino Acid Content

Total nitrogen was determined by weighing ˜0.5 g of the steep-water in vario MAX CN elemental N analyzer (Elementar Analysensysteme GmbH, Donaustrabe, Germany). The standard used contained 2000 ppm N. The samples were run in duplicates and the average is reported below.

Free amino nitrogen (FAN) was assayed by mixing 5-20 μL of steep-water with 2 ml of deionized water and 1 mL of Ninhydrin solution (6.5% of Na₂HPO₄, 6% KH₂PO₄, 0.5% ninhydrin, and 0.3% fructose). The mixture was then capped tightly and boiled for 16 minutes. After boiling the mixture was allowed to cool to room temperature and 5 mL of diluent (0.2% KIO₃, 60% deionized water, and 40% ethanol, (190 proof)) was added. The color was allowed to develop and was measured at 570 nm. A glycine standard of 2-ppm nitrogen was used as the control in each assay.

The results provided in Table 1.A show that endogenous protease activity was highest, as judging by the increased ratio of free amino nitrogen to total nitrogen, in cultures with pH control by the addition of 1% CaCO₃. Hence, an enhanced feed stock with increased FAN concentration can be made by maintaining the pH at greater than 5.0, 5.2, 5.3, or 5.4. Anaerobic conditions also favored the endogenous protease activity over aerobic conditions at all temperatures tested.

TABLE 1.A
FAN/N (Free ammo nitrogen to total nitrogen ratio)

| | Time (h) | | | |
|---|---|---|---|---|
| | 0 | 24 | 48 | 72 |
| 30° C. aerobic | 18.3% | 19.3% | 18.9% | 23.9% |
| 30° C. aerobic CaCO₃ | 19.2% | 18.7% | 23.0% | 26.5% |
| 30° C. anaerobic | 18.7% | 17.6% | 21.1% | 23.6% |
| 30° C. anaerobic CaCO₃ | 18.7% | 19.2% | 24.1% | 33.4% |
| 38° C. aerobic | 18.2% | 20.3% | 24.4% | 24.8% |
| 38° C. aerobic CaCO₃ | 19.2% | 24.3% | 29.6% | 29.4% |
| 38° C. anaerobic | 18.8% | 19.3% | 25.3% | 28.0% |
| 38° C. anaerobic CaCO₃ | 18.8% | 23.1% | 31.3% | 39.2% |
| 49° C. aerobic | 18.7% | 19.2% | 19.4% | 22.5% |
| 49° C. aerobic CaCO₃ | 17.1% | 18.7% | 20.0% | 21.2% |
| 49° C. anaerobic | 17.1% | 15.3% | 19.8% | 21.8% |
| 49° C. anaerobic CaCO₃ | 18.4% | 19.6% | 20.9% | 23.9% |

Additional assays to determine lysine and methionine contents were conducted using the USDA 6.011 method. Most of the samples showed that the content of lysine in total nitrogen increased in steep-water incubated aerobically at all temperatures with or without pH control. However, the highest lysine to total nitrogen ratio was observed in steep-water incubated at 30° C. anaerobically with pH control. The same condition also generated a steep-water high in methionine content, both as ratio to total nitrogen and in concentration as is in steep-water. Hence, an enhanced steep-water with higher concentrations of lysine and/or methionine can be made by controlling incubation temperatures at from about 25° C. to about 40° C., or from about 27° C. to about 35° C.

TABLE 1.B

| T72 | | | T72 | |
|---|---|---|---|---|
| Lysine/total protein | | | Methionine/total protein | |
| | w/o CaCO₃ | With CaCO₃ | w/o CaCO₃ | With CaCO₃ |
| Anaerobic | | | | |
| 47° C. | 4.6% | 2.2% | 1.3% | 1.1% |
| 35° C. | 5.3% | 2.5% | 2.1% | 1.7% |
| 30° C. | 4.1% | 10.2% | 1.0% | 4.0% |
| Aerobic | | | | |
| 47° C. | 2.6% | 8.3% | 0.5% | 2.2% |
| 35° C. | 7.4% | 5.7% | 1.9% | 0.6% |
| 30° C. | 7.3% | 8.5% | 2.1% | 1.9% |

Additional studies were done to characterize yeast extract produced by incubating yeast in steep water. The experiments described herein involved adding endogenous yeast to steep-water, but it is expected that the same results would be achieved if steep-water had been incubated under the conditions provided above which yield increased yeast populations without the addition of exogenous yeast.

Baker's yeast (Saccharomyces cerevisiae) and Brewer's yeast (Saccharomyces cerevisiae) pastes were obtained from various Cargill facilities and added to either water or light steep-water to a final dry cell weight of 13.8%. Autolysis was accomplished by adjusting the pH to 5.0 and the temperature 45° C. and incubating for between 24-48 hours with 150 rpm mixing. During the autolysis incubation, residual sugars in steep-water (1.1% glucose and 0.7% fructose) were utilized by Baker's or Brewer's yeasts. After autolysis, insoluble yeast materials were separated from supernatant by centrifugation at 8,000 rpm (4500-×g) for 20 minutes at 10° C. The yield of supernatant was calculated as the percent of solubilized nitrogen to total nitrogen after 48 hours of incubation and is shown in the following Table 1.C. The degree of protein hydrolysis was compared using the ratio of free amino nitrogen (FAN) to total nitrogen (N).

TABLE 1.C

| (%) | |
|---|---|
| Experiment A | Experiment B |

| | Yield | FAN/N | Yield | FAN/N |
|---|---|---|---|---|
| Baker's yeast in water | 68.1 | 31.6 | 65.2 | 37 |
| Baker's yeast in LSW | 63 | 38.9 | 56.1 | 41.4 |
| Brewer's yeast in water | 66.2 | 32.9 | 59.1 | 41 |
| Brewer's yeast in LSW | 61.9 | 32.1 | 50.8 | 35.4 |

The yeast autolysates in water or in steep-water were used in a model ethanol fermentation containing 0.1% KH₂PO₄, 0.04% MgSO₄.7H₂O, 10% glucose, and 1.25% protein, pH=5.0. A commercial yeast extract obtained from BD Diagnostics (Sparks, Md.) was used as a control in this fermentation. BD yeast extract has a FAN/N of 32.5%. 1.25% yeast culture pre-grown overnight in YPD (5 g/L yeast extract, 5 g/L peptone, 2 g/L KH2PO₄, and 0.4 g/L MgSO₄.7H₂O, pH=5) medium was used to inoculate each of the cultural media containing various types of yeast autolysates. The cultures were in duplicates and incubated at 30° C. with 100 rpm shaking. Higher biomass was found in cultures containing yeast/steep-water autolysate than that of commercial yeast extract. The results are shown in the following table. Biomass was measured spectroscopically at 600 nm after proper dilution in water. Glucose concentration was measured by HPLC in g/L as is basis. The ethanol yields of all these cultures are 49%+/−0.5%.

TABLE 1.D

| | T0 | | | T5 | | | T11 | | |
|---|---|---|---|---|---|---|---|---|---|
| | OD | pH | Glu | OD | pH | Glu | OD | pH | Glu |
| BD YE | 4 | 5 | 100 | 38 | 4.3 | 75 | 82 | 4.7 | 0 |
| Baker's | 3.9 | 5 | 100 | 34 | 4.3 | 74 | 81 | 4.6 | 0 |
| yeast/water | | | | | | | | | |
| Baker's | 4.8 | 5 | 97 | 35 | 4.5 | 75 | 101 | 4.5 | 0 |
| yeast/steep | | | | | | | | | |
| Brewer's | 4.2 | 5 | 100 | 35 | 4.6 | 75 | 110 | 4.5 | 0 |
| yeast/water | | | | | | | | | |
| Brewer's | 4.2 | 5 | 97 | 37 | 4.5 | 75 | 115 | 4.4 | 0 |
| yeast/steep | | | | | | | | | |

B Sugar Assay

Glucose concentration was determined by diluting steep-water 1:1 with eluent (0.001 N H₂SO₄ and 0.00001% NaN₃), and filtering through a 0.45μ filter. Ten μL samples were injected into a Biorad Aminex HPX-87H column and eluted at 60° C. with the above eluent. Standards for maltotriose, maltose, citric, glucose, fructose, succinic, lactic, acetic, propionic, and ethanol were used as controls.

Glucose disappeared fast at 30° C. and 38° C. No glucose depletion was observed at 49° C. under anaerobic conditions. Hence, to make an enhanced steep-water with decreased concentrations of glucose the steep-water can be incubated at temperatures less than 49° C.

TABLE 1.B
Glucose concentration in steep-water in g/L

Time hr

| | 0 | 24 | 48 | 72 |
|---|---|---|---|---|
| 30° C. aerobic | 9.432 | 0.112 | 0.088 | 0.072 |
| 30° C. aerobic CaCO₃ | 9.072 | 0.094 | 0.114 | 0.11 |
| 30° C. anaerobic | 9.3 | 0.094 | 0.072 | 0.048 |
| 30° C. anaerobic CaCO₃ | 9.072 | 0.078 | 0.128 | 0.092 |
| 38° C. aerobic | 9.302 | 0.018 | 0.064 | 0.038 |
| 38° C. aerobic CaCO₃ | 8.966 | 0.062 | 0.096 | 0.094 |
| 38° C. anaerobic | 9.292 | 0.062 | 0.06 | 0.046 |
| 38° C. anaerobic CaCO₃ | 9.008 | 0.108 | 0.084 | 0.064 |
| 49° C. aerobic | 9.326 | 9.042 | 9.358 | 9.902 |
| 49° C. aerobic CaCO₃ | 9.026 | 6.516 | 0.344 | 0.35 |
| 49° C. anaerobic | 9.286 | 9.38 | 9.168 | 9.119 |
| 49° C. anaerobic CaCO₃ | 8.986 | 8.504 | 2.93 | 2.834 |

C. Acetate

Organic acids were assayed using the same procedure as described above for sugars. High acetate concentration was found at 38° C. aerobically without pH control. SO₂ seemed to further enhanced acetate production. Up to 11.5 g/L acetate was found after 72 hours of incubation. Hence, enhanced steep-water with high acetate content can be made by incubating the steep-water at from about 30° C. to about 46° C., or from about 30° C. to about 40° C. under aerobic conditions.

TABLE 1.C
Acetate concentration in g/L in steep-water

| | 0 | 24 | 48 | 72 |
|---|---|---|---|---|
| 30° C. aerobic | 0.104 | 1.052 | 6.124 | 6.366 |
| 30° C. aerobic CaCO₃ | 0.090 | 1.280 | 6.384 | 8.652 |
| 30° C. anaerobic | 0.098 | 0.152 | 0.292 | 0.514 |
| 30° C. anaerobic CaCO₃ | 0.096 | 0.604 | 1.681 | 3.026 |
| 38° C. aerobic | 0.092 | 3.432 | 8.924 | 11.478 |
| 38° C. aerobic CaCO₃ | 0.098 | 3.866 | 8.200 | 10.146 |
| 38° C. anaerobic | 0.098 | 0.358 | 1.052 | 1.906 |
| 38° C. anaerobic CaCO₃ | 0.090 | 1.658 | 3.286 | 4.174 |
| 49° C. aerobic | 0.104 | 0.200 | 0.144 | 0.178 |
| 49° C. aerobic CaCO₃ | 0.092 | 0.612 | 5.358 | 6.754 |
| 49° C. anaerobic | 0.090 | 0.118 | 0.118 | 0.116 |
| 49° C. anaerobic CaCO₃ | 0.094 | 0.156 | 0.464 | 0.510 |

D. Lactate

High lactate concentrations were obtained under anaerobic conditions, and successively more lactate was produced as temperature was increased, with the highest concentration being found at 49° C. with pH control (pH 5.4). Lactate was consumed fastest under aerobic conditions at 30° C. Hence, enhanced steep-water with elevated lactate concentrations can be made by incubating light steep-water under anaerobic conditions at temperatures at from about 36° C. to about 55° C., or from about 38° C. to about 52° C. It is expected that additional lactate could be made by adding more glucose to the enhanced steep-water.

TABLE 1.D
Lactate concentration in steep-water in g/L

| | 0 | 24 | 48 | 72 |
|---|---|---|---|---|
| 30° C. aerobic | 18.038 | 15.592 | 8.458 | 1.298 |
| 30° C. aerobic CaCO₃ | 17.066 | 17.842 | 16.824 | 7.684 |
| 30° C. anaerobic | 17.394 | 17.904 | 18.424 | 19.156 |
| 30° C. anaerobic CaCO₃ | 17.14 | 19.686 | 21.146 | 18.850 |
| 38° C. aerobic | 17.716 | 15.120 | 8.594 | 1.038 |
| 38° C. aerobic CaCO₃ | 16.906 | 21.434 | 11.552 | 2.560 |
| 38° C. anaerobic | 17.746 | 20.002 | 22.396 | 20.474 |
| 38° C. anaerobic CaCO₃ | 16.976 | 24.030 | 22.120 | 21.046 |
| 49° C. aerobic | 17.824 | 18.838 | 18.504 | 19.780 |
| 49° C. aerobic CaCO₃ | 16.958 | 21.016 | 17.446 | 14.586 |
| 49° C. anaerobic | 17.72 | 18.130 | 18.178 | 18.208 |
| 49° C. anaerobic CaCO₃ | 16.58 | 17.626 | 23.316 | 23.534 |

Additionally, high succinate concentration was found at 35° C. under anaerobic conditions with pH control. Approximately, 3 g/L of succinate was made under these conditions along with high lactate production.

E. Ethanol

The method used to measure ethanol concentration was the same as that used for measuring sugars. Ethanol was most concentrated in steep-water cultures incubated under anaerobic conditions at 30° C. and 38° C., without pH control. Ethanol disappeared completely at 30° C. under aerobic conditions.

TABLE 1.E
Ethanol g/L

| | 0 | 24 | 48 | 72 |
|---|---|---|---|---|
| 30° C. aerobic | 1.656 | 6.810 | 0 | 0.000 |
| 30° C. aerobic CaCO₃ | 1.682 | 5.424 | 0 | 0.000 |
| 30° C. anaerobic | 1.572 | 8.746 | 8.534 | 8.530 |
| 30° C. anaerobic CaCO₃ | 1.66 | 6.592 | 8.04 | 9.214 |
| 38° C. aerobic | 1.614 | 3.714 | 0.334 | 0.408 |
| 38° C. aerobic CaCO₃ | 1.662 | 2.200 | 0.66 | 0.994 |
| 38° C. anaerobic | 1.564 | 7.334 | 7.682 | 7.948 |
| 38° C. anaerobic CaCO₃ | 1.648 | 4.622 | 6.072 | 6.578 |
| 49° C. aerobic | 1.568 | 1.510 | 1.294 | 0.744 |
| 49° C. aerobic CaCO₃ | 1.648 | 1.416 | 0.914 | 0.664 |
| 49° C. anaerobic | 1.596 | 1.650 | 1.654 | 1.658 |
| 49° C. anaerobic CaCO₃ | 1.656 | 1.592 | 1.660 | 1.656 |

F. Biotin

The dry solids (DS) value for CSL+ was determined by AACC44-40 method. Biotin content in steep-water was determined using the AOAC 960.46 method. The results indicate that biotin enhanced steep-water can be made by incubating the sample at 38° C. or 30° C. under aerobic conditions. Biotin levels were also observed to be higher when the steep-water was inoculated with Baker's yeast.

TABLE 1.F
Biotin concentration in steep-water
in ug/g of steep-water dry solid

| | T72 | |
|---|---|---|
| | w/o CaCO₃ | With CaCO₃ |
| Anaerobic | | |
| 47° C. | 0.6 | 0.49 |
| 38° C. | 0.55 | 0.48 |
| 30° C. | 0.94 | 0.76 |
| Baker's yeast/30° C. | 1.01 | |
| Aerobic | | |
| 47° C. | 0.8 | 0.5 |
| 38° C. | 1.19 | 1.11 |
| 30° C. | 1.28 | 0.7 |
| Baker's yeast/30° C. | 0.91 | |

### Example 2

### Enrichment of Bacteriocin Producers in Steep-Water

Lactic acid bacteria and other bacteria may be enriched, or exogenously applied and/or enriched by incubating light or concentrated steep-water samples at various temperatures such as room temperature, 30° C., 37° C. and/or 42° C., to encourage the growth of specific bacteriocin producing bacteria (Contreras et. al., 1997—Appl. Environ Microbiol, January, 63(1): 13-20). Factors such as temperature, pH, water activity, redox potential and the presence of inhibitory compounds in the various corn steep-water samples will determine bacterial growth. The bacteriocins produced may be identified using established analytical procedures. In addition steep-water samples may be supplemented with nutrients to support the growth of the bacteriocin-producing microorganism. Depending on the incubation times being employed, nutrient depletion will be compensated for by the addition of fresh medium. Limitation of specific essential molecules needed for cell metabolism, amino acids, vitamins, minerals and etc, may decrease the growth rate. Fed-batch fermentation is a viable method to obtain high concentrations of industrially significant bacteriocins from bacteria.

### Example 3

### Combination of Propionic Acid and Bacteriocins

Similar to example 2, except low phosphorous steep-water (LPCSL) will be used to enrich for propionic acid bacteria instead of lactic acid bacteria. The propionic acid bacteria produce some bacteriocins and are useful in preventing spoilage in dairy products and other foods. The elevated levels of propionic acid would be effective in preventing fungal growth in grains, non-grain feed ingredients, complete feeds and feed products. Steep-water with increased propionic acid content as well as bacteriocins will have the benefits of bacteriocin as well as anti-fungal properties conferred by propionic acid.

### Example 4

### Fermentation with Endogenous Microorganisms to Produce Vitamin B₁₂

Low phosphorous light steep-water (pH 6.0) is incubated at 32° C. for 144 h to allow for growth of endogenous microorganisms that produce propionic acid and vitamin B₁₂. Approximately 15 g/L lactate, 10 g/L dextrose and 5 g/L fructose are converted to propionic acid at yields near 50% of the initial substrate concentrations and to 5 mg/L vitamin B₁₂. The resulting steep-water with increased B₁₂ is blended with other feed ingredients to make an animal feed enriched with vitamin B₁₂, or alternatively used as a fermentation feedstock for a B₁₂ requiring fermentation.

### Example 5

### Fermentation with Bacillus

Same as Example 1 but rather than relying solely on the indigenous Bacillus population, the steep-water is inoculated with Bacillus spp. to produce protease.

### Example 6

### Fermentation with Aspergillus

Same as example 1 but the steep-water is incubated at 30-35° C. to allow the indigenous Aspergillus spp. to grow, or alternately inoculated with exogenous Aspergillus spp. In the latter instance, very high aeration and agitation is necessary due to the viscosity imparted by the growing fungal mycelium. Phytase activity in the enzyme-enriched corn steep-water is assayed using established analytical procedures.

### Example 7

### Incubation with Lysine-Overproducing Mutants of Corynebacterium or Brevibacterium spp.

Same as example 1 except low phosphorous steep-water is inoculated with lysine-overproducing mutants of Corynebacterium or Brevibacterium spp.

### Example 8

### Incubation with Methionine-Overproducing Mutants of Corynebacterium or Brevibacterium spp.

Same as example 1 except low phosphorous steep-water is inoculated with methionine-overproducing mutants of Corynebacterium or Brevibacterium spp.

Numerous modifications and variations in practice of the invention are expected to occur to those skilled in the art upon consideration of the foregoing detailed description of the invention. Consequently, such modifications and variations are intended to be included within the scope of the following claims.

1-26. (canceled)

27. A method of making a feed additive or a feedstock comprising:
providing corn steep-water having a solids content of from about 8% to about 50% on a dry weight basis;
fermenting the steep-water in the presence of at least one microbe to generate fermented steep-water,
drying the fermented steep-water to generate an enhanced steep-water having a solids content of from about 30% to about 90% on a dry weight basis, thereby making said feed additive or said feedstock.

28. The method of claim 27 wherein the corn steep-water is a low phosphorous steep-water.

29. The method of claim 28 wherein the low phosphorous steep-water is a low phytate steep-water.

30. The method of claim 27 wherein the at least one microbe is endogenous to the steep-water.

31. The method of claim 27 wherein the at least one microbe is exogenous to the steep-water.

32. The method of claim 27, wherein said at least one microbe is an organic acid-producing microbe, wherein said fermenting is under conditions that increase organic acid content at least about 10% on a dry weight basis.

33. The method of claim 32, wherein said organic acid-producing microbe is selected from the group consisting of a lactic acid-producing microbe, a propionic acid-producing microbe, a citric acid-producing microbe, a fumaric acid-producing microbe, a succinic acid-producing microbe, a formic acid-producing microbe, a benzoic acid-producing microbe, and an acetic acid-producing microbe.

34. The method of claim 33 wherein said organic acid-producing microbe is selected from the group consisting of Lactobacillus spp., Lactococcus spp., Leuconostoc spp., Bacillus coagulans, Propionibacterium acidipropionici, Propionibacterium freudenreichii, Propionibacterium shermanii, Propionibacterium jensenii, and Propionibacterium thoenii.

35. The method of claim 32 wherein said fermenting is performed at a temperature of from about 36° C. to about 55° C. under anaerobic conditions.

36. The method of claim 35 wherein the fermenting is performed at a pH of from about 4.5 to about 6.0.

37. The method of claim 32 wherein the fermented or enhanced steep-water comprises a lactate concentration of at least about 180 g lactate per Kg of steep-water solids.

38. The method of claim 27 wherein said at least one microbe is a direct-fed microbial (DFM), wherein said fermenting is under conditions that increase the numbers of said DFM.

39. The method of claim 38 wherein said DFM is selected from the group consisting of Lactobacillus plantrum, Lactobacillus casei, Lactobacillus acidophilus, Enterococcus faecium, Bifidobacterium bifidium, Bifidobacterium thermophilum, Bifidobacterium longum, Bacillus subtillis, Saccharomyces cerevisiae, Aspergillus oryzae and mixtures thereof.

40. The method of claim 38, wherein said fermenting is performed at a temperature of at least 25° C. for at least about 10 hours.

41. The method of claim 27 wherein said fermenting is under conditions that induce said at least one microbe to produce or increase production of one or more enzymes.

42. The method of claim 41 wherein said at least one microbe is selected from the group consisting of Bacillus amyloliquefaciens, Bacillus licheniformis, Bacillus subtilis, Aspergillus niger, Aspergillus oryzae, Bacillus lentus, Bacillus stearothermophilus, Humicola insolens, Trichoderma longibranchiatum, Aspergillus niveus and mixtures thereof.

43. The method of claim 41 wherein the fermenting is performed at a temperature of at least about 28° C. for about 24 hours to about 72 hours.

44. The method of claim 41 wherein the enzyme is selected from the group consisting of a protease, a xylane, an amylase, and a phytase.

45. The method of claim 27 wherein said microbe is yeast, wherein said fermenting is under conditions that generate a enhanced steep-water having at least about 70% yeast on a dry weight basis.

46. The method of claim 45 wherein said yeast is selected from the group consisting of Saccharomyces cerevisiae, Candida utilis, Kluyveromyces marxianus, and Torulaspora delbrueckii.

47. The method of claim 45 wherein the fermenting is performed at a temperature of from about 25° C. to about 45° C. under aerobic conditions.

48. The method of claim 45 wherein the fermenting is performed at a temperature of at least about 28° C. for at least about 24 hours.

49. The method of claim 45 wherein the fermented steep-water comprises at least 7 g acetate per L of fermented steep-water.

50. The method of claim 45 wherein the fermented or enhanced steep-water comprises at least about 1 μg biotin per g of steep-water solids.

51. The method of claim 45 further comprising lysing, at least partially, said at least one yeast in the fermented or enhanced steep-water to generate a yeast extract.

52. The method of claim 51 wherein said lysing comprises incubating the fermented or enhanced steep-water at a pH of from about 4.7 to about 5.2 and a temperature of from about 42° C. to about 48° C.

53. The method of claim 51 wherein at least some of said lysing is autolysing by said at least one yeast.

54. The method of claim 53 wherein the autolysing is performed under aerobic conditions at a pH of about 5.0 and a temperature of about 45° C.

55. A yeast extract made by the method of claim 51.

56. The method of claim 27 wherein said fermenting is under conditions that induce said at least one microbe to produce or increase production of one or more vitamins.

57. The method of claim 56 wherein said microbe is selected from the group consisting of Propionibacterium shermannii, Ashbya gossypii, Eremothecium ashbyii, Bacillus spp., Gluconobacter oxidans subsp. suboxidans, Serratia marcescens, Pseudomonas denitrificans, Mortierella alpine and combinations thereof.

58. The method of claim 56 wherein said vitamin is B₁₂, riboflavin, arachidonic acid, linolenic acid, vitamin C, biotin, mead acid, pantothenate, or thiamine.

59. The method of claim 56 wherein said fermenting is performed at a temperature of at least about 20° C. for at least about 10 hours.

60. The method of claim 56 wherein said fermented or enhanced steep-water comprises an increase of at least about 400 μg vitamin/Kg steep-water solids.

61. The method of claim 27 wherein said fermentation is under conditions that induce said at least one microbe to produce or increase production of free amino nitrogen (FAN) or one or more amino-acids.

62. The method of claim 61 wherein said microbe is selected from the group consisting of Bacullus subtilis, Bacillus amyloliquefaciens, Aspergillus niger, Aspergillus oryzae, Corynebacterium glutamicum, Corynebacterium acetoacidophilum, Brevibacterium lactofermentum, Brevibacterium flavum, Brevibacterium divaricatum, and Corynebacterium lilium.

63. The method of claim 61 wherein said amino acid is lysine or methionine.

64. The method of claim 61 wherein said fermenting is performed at a temperature of at least about 28° C. for about 24 to about 72 hours.

65. The method of claim 61 wherein said fermented or enhanced steep-water comprises at least about 5 g FAN per Kg steep-water solids.

66. The method of claim 61 wherein said fermenting is performed at a temperature of at least about 30° C. for at least about 48 hours.

67. The method of claim 63 wherein said fermented or enhanced steep-water comprises at least about 20 g lysine or 4 g methionine per Kg steep-water solids.

68. The method of claim 27 wherein the enhanced steep-water has a solids content of from about 40% to about 80% on a dry weight basis.

69. The method of claim 27 wherein the enhanced steep-water has a solids content of from about 50% to about 70% on a dry weight basis.

70. The method of claim 27 wherein the enhanced steep-water has a solids content of from about 60% to about 75% on a dry weight basis.

71. The method of claim 27 further comprising removing, at least partially, the at least one microbe from the fermented or enhanced steep-water.

72. The method of claim 71 further comprising mixing the removed at least one microbe with the feed additive or feedstock.

73. The method of claim 27 further comprising mixing the feed additive or feedstock with a feedstuff to generate an animal feed having a moisture content of at least about 12% by weight.

74. A feed additive or feedstock produced by the method of claim 27.

75. An animal feed comprising a feed additive or feedstock produced by the method of claim 27.
